(19) United States
(12) Patent Application Publication
(10) Pub. No.: US 2019/0000402 A1
(43) Pub. Date: Jan. 3, 2019

(54) Human Body Condition and Behavior Monitoring Alarm System

(71) Applicant: Haiyun Kuang, Fuzhou, Fujian (CN)

(72) Inventor: Minghong Tang, Fuzhou, Fujian (CN)

(73) Assignees: Haiyun Kuang, Fuzhou, Fujian (CN); Haiyun Kuang, Fuzhou, Fujian (CN)

(21) Appl. No.: 16/066,306

(22) PCT Filed: Feb. 22, 2017
(86) PCT No.: PCT/CN2017/074385

§ 371 Date: Jun. 27, 2018

(30) Foreign Application Priority Data
Mar. 2, 2016 (CN) 201620159214.8

(51) Int. Cl. A61B 5/00; G08B 21/04; G08B 25/10; A61B 5/01; A61B 5/0205; A61B 5/0402; A61B 5/11; A61B 7/04; G01N 33/497

(52) U.S. Cl. CPC A61B 5/746; A61B 5/021; G08B 25/10; A61B 5/01; A61B 5/02055; A61B 5/0402; A61B 5/1116; A61B 5/0006; A61B 5/0008; A61B 5/741; A61B 5/7435; A61B 5/1112; A61B 7/04; A61B 5/6804; A61B 5/6843; G01N 33/497; G08B 21/0446; G08B 21/0453

(57) ABSTRACT

The utility model relates to the field of smart wearable devices, in particular to a human body condition and behavior monitoring alarm system. The human body condition and behavior monitoring alarm system comprises a master chip, a display, a temperature and humidity sensor, a blood pressure sensor, a brain wave sensor, an electronic gyroscope, a heart rate sensor, a pressure sensor, a hydrogen sulfide sensor, a noise detection module, a Bluetooth module and a power supply module. The human body condition and behavior monitoring alarm system is capable of monitoring both physical health conditions and behavior conditions of human bodies.

### TECHNICAL FIELD

The utility model relates to the field of smart wearable devices, in particular to a human body condition and behavior monitoring alarm system.

### DESCRIPTION OF RELATED ART

With the advance of the social technology, more and more smart wearable devices have entered people's life. Modern people pay more and more attention to physical health, and following the current research and development tide of smart wear, more and more researchers put research and development emphasis into the field of smart wear for health. For instance, Chinese Invention Patent 201110371533.7 titled ‘Heart Rate Warning System’ discloses a smart garment with a heart rate warning function, which can detect the psychological condition of the wearer in real time and send out a prompt alarm in time when the heart rate of the wearer is abnormal.

With the development of society, besides the requirement for heart rate monitoring, people also have the requirement for more comprehensively monitoring of various physical conditions and even behavior conditions, such as abnormal behaviors including excited emotions, physical conflicts and falling of bodies so as to know and handle these conditions as early as possible.

However, at present, only devices for monitoring physical conditions have been developed by those skilled in the field of smart wear, and smart wearable devices capable of monitoring behavior conditions of human bodies are not available yet.

### TECHNICAL PROBLEMS

The objective of the utility model is to provide a human body condition and behavior monitoring alarm system, which is capable of monitoring both physical conditions and behavior conditions of human bodies.

### SOLUTION TO PROBLEMS

### Technical Solution

The utility model is realized through the following technical scheme: a human body condition and behavior monitoring alarm system is characterized by:

comprising:

a master chip used for receiving, sending and processing information and sending alarm commands;

a display connected with the master chip and used for displaying information data;

a temperature and humidity sensor connected with the master chip and used for sensing and collecting human body temperature and humidity data in real time and transmitting the collected data to the master chip;

a blood pressure sensor connected with the master chip and used for sensing and collecting human body blood pressure data in real time and transmitting the collected data to the master chip;

a brain wave sensor connected with the master chip and used for sensing and collecting human body brain wave data in real time and transmitting the collected data to the master chip;

an electronic gyroscope connected with the master chip and used for sensing and collecting human body posture data in real time and transmitting the collected data to the master chip;

a heart rate sensor connected with the master chip and used for sensing and collecting human body heart rate data in real time and transmitting the collected data to the master chip;

a pressure sensor connected with the master chip and used for sensing and collecting human body weight data as well as pressure data of a garment a human body in real time and transmitting the collected data to the master chip;

a hydrogen sulfide sensor connected with the master chip and used for sensing and collecting concentration data of hydrogen sulfide discharged by the human body in real time and transmitting the collected data to the master chip;

a noise detection module connected with the master chip and used for monitoring the loudness of noise in the ambient environment;

a Bluetooth module connected with the master chip and used for sending data and alarm information; and

a power supply module connected with the master chip and used for providing a system power supply.

The master chip is further connected with a voice alarm module used for sending out an alarm signal. When the human body temperature and humidity data, the human body blood pressure data, the human body brain wave data, the human body heart rate data, the human body posture data and the hydrogen sulfide concentration data received by the master chip exceed set values, the master chip instantly instructs the voice alarm module to send out an alarm signal.

The master chip is further connected with a recording module used for recording sounds in the ambient environment. When detecting that the noise, sensed by the noise detection module, in the ambient environment is greater than a set value, the master chip instantly controls the recording module to record the noise.

The operating principle of the human body condition and behavior monitoring alarm system is as follows:

The human body condition and behavior monitoring alarm system of the utility model detects the health condition of the human body in the following way:

All the sensors transmit the human body temperature and humidity data, the human body blood pressure data, the human body brain wave data, the human body posture data and other data to the master chip in real time, the master chip compares all the sets of data with human body pathological data indicators set and stored in the master chip and instantly instructs the voice alarm module to send out an alarm signal once finding that the data transmitted from the sensors are abnormal, and at this moment, the alarm information is sent to an external receiving terminal through a Bluetooth, WIFI or GSM module so that the wearer and relevant personnel can know an abnormal physical condition in time.

Through noise monitoring, when increased suddenly, surrounding noise is recorded and transmitted to relevant personnel, and the relevant personnel can know the current condition, such as quarrels and physical conflicts with other people, of the wearer by playing the record back.

The master chip can automatically figure out whether or not the posture of the wearer is normal by monitoring changes of the human body posture data in real time and instantly instructs the voice alarm module to send out an alarm signal once realizing an abnormal condition (such as sudden falling), and at this moment, the alarm information is sent to the external receiving terminal through a Bluetooth, WIFI and wireless communication module so that the wearer and relevant personnel can know the abnormal behavior condition in time.

Herein, the brain wave sensor is generally disposed in a cap; the hydrogen sulfide sensor is generally disposed on a pair of trousers and used for detecting whether or not the wearer relieve himself/herself so that relevant personnel can realize and handle the defecation condition in time; and two pressure sensors are adopted generally, one pressure sensor is disposed in a shoe sole and used for measuring the weight of the human body, the other pressure sensor is disposed in the shoulder part of the garment, and the master chip can figure out the thickness of the garment worn by the wearer according to the pressure sensed by the pressure sensor on the shoulder part of the garment and then automatically amplifies the volume of the speaker when the garment is thick.

To prevent the sensors from being wetted by water when the garment is washed, connection circuits of the sensors are all embedded in an interlayer of the garment, and the joints between the connection circuits and the master chip are of a waterproof structure.

In order to better implement the scheme, the following optimization scheme is further provided:

Furthermore, the human body condition and behavior monitoring alarm system further comprises a GSM module connected with the master chip and used for sending alarm information to a mobile phone.

The GSM module is provided with an SIM card holder, a GPRS module and a GPS positioning module.

Furthermore, the human body condition and behavior monitoring alarm system further comprises a WIFI module connected with the master chip and used for sending alarm information to an external routing device.

Furthermore, the human body condition and behavior monitoring alarm system further comprises a touch screen connected with the master chip and used for operating the system.

Furthermore, the human body condition and behavior monitoring alarm system further comprises an electronic stethoscope connected with the recording module and used for acquiring human body sound data.

The human body sound data acquired by the electronic stethoscope are transmitted to the recording module so as to be recorded.

A sound hole of the electronic stethoscope is generally disposed at a position, close to the chest, of the inner side of the garment so that abnormal human body sound data of the wearer can be found in time.

Furthermore, the master chip is an STM32 master chip.

Furthermore, the display is an OLED display screen.

Furthermore, the temperature and humidity sensor, the blood pressure sensor, the brain wave sensor, the electronic gyroscope, the heart rate sensor, the pressure sensor and the hydrogen sulfide sensor are all sleeved with waterproof shells.

Furthermore, the human body condition and behavior monitoring alarm system further comprises a camera connected with the master chip and used for recording images of the ambient environment.

The monitor can know the condition around the wearer through the images recorded by the camera, and the camera is generally disposed at a position corresponding to the chest or on a neckline.

Furthermore, the noise detection module is provided with a microphone.

The microphone is used for acquiring sounds around the wearer and can be disposed in the garment, on the neckline or at any position corresponding to the chest.

Furthermore, the voice alarm module is provided with a speaker.

The speaker can be used for playing voice alarm information and also can be used for playing key sounds or other sounds in the system.

Furthermore, the human body condition and behavior monitoring alarm system further comprises a solar cell connected with the master chip and used for providing a system power supply.

Furthermore, the human body condition and behavior monitoring alarm system further comprises a radio module 23 connected with the master chip 1.

### BENEFICIAL EFFECTS OF THE INVENTION

### Beneficial Effects

Compared with the prior art, the utility model has the following beneficial effect:

First, through comparison of the data collected by various sensors of the utility model, abnormal body conditions of the wearer can be realized in time, and alarm information can be sent out in time.

Second, through the means including sound recording, video recording, photographing, posture detection and the like, the behavior condition of the wearer can be well monitored, and in-time intervention can be conducted when an abnormal behavior occurs.

Third, the human body condition and behavior monitoring alarm system of the utility model is convenient to wear, has rich functions and is suitable for users at different ages.

### BRIEF DESCRIPTION OF DRAWINGS

### Description of Drawings

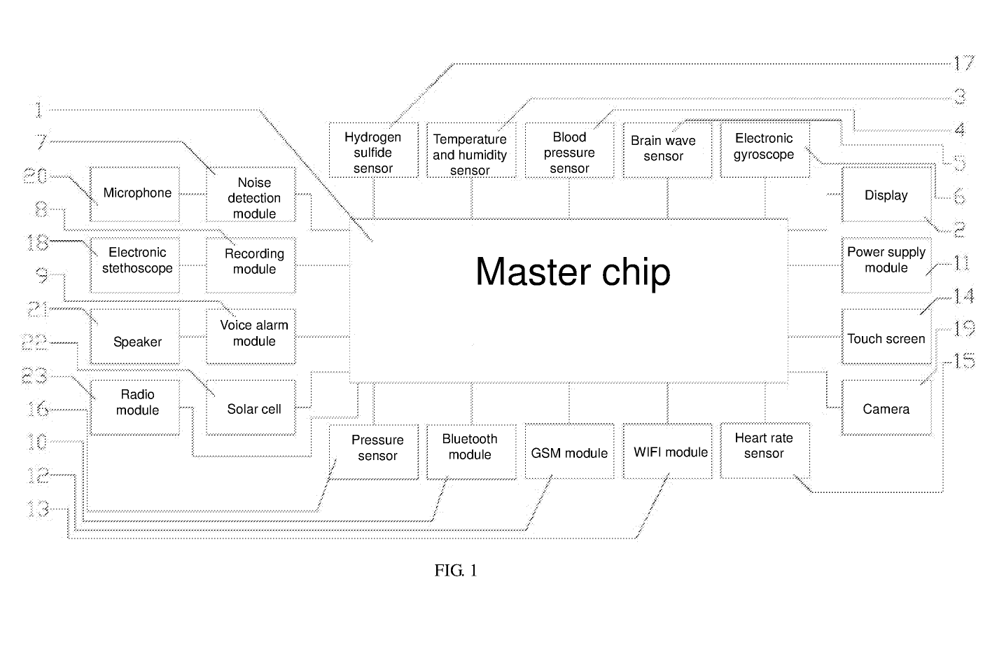

FIG. 1 is a block diagram of modules of the utility model;

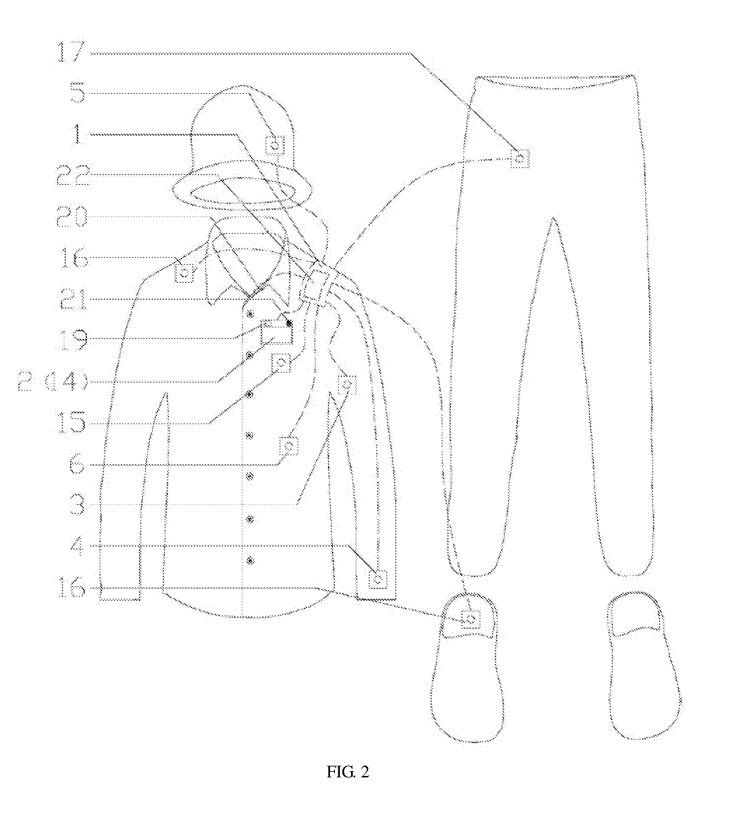

FIG. 2 is a use diagram of the utility model.

Description of Reference Signs:

1-master chip, 2-display, 3-temperature and humidity sensor, 4-blood pressure sensor, 5-brain wave sensor, 6-electronic gyroscope, 7-noise detection module, 8-recording module, 9-voice alarm module, 10-Bluetooth module, 11-power supply module, 12-GSM module, 13-WIFI module, 14-touch screen, 15-heart rate sensor, 16-pressure sensor, 17-hydrogen sulfide sensor, 18-electronic stethoscope, 19-camera, 20-microphone, 21-speaker, 22-solar cell, 23-radio module.

### EMBODIMENTS OF THE INVENTION

### Detailed Description of the Invention

A detailed description of the utility model is given with the accompanying drawings as follows:

As is shown in FIGS. 1-2, a human body condition and behavior monitoring alarm system of the utility model comprises:

a master chip 1 used for receiving, sending and processing information and sending alarm commands;

a display 2 connected with the master chip 1 and used for displaying information data;

a temperature and humidity sensor 3 connected with the master chip 1 and used for sensing and collecting human body temperature and humidity data in real time and transmitting the collected data to the master chip 1;

a blood pressure sensor 4 connected with the master chip 1 and used for sensing and collecting human body blood pressure data in real time and transmitting the collected data to the master chip 1;

a brain wave sensor 5 connected with the master chip 1 and used for sensing and collecting human body brain wave data in real time and transmitting the collected data to the master chip 1;

an electronic gyroscope 6 connected with the master chip 1 and used for sensing and collecting human body posture data in real time and transmitting the collected data to the master chip 1;

a heart rate sensor 15 connected with the master chip 1 and used for sensing and collecting human body heart rate data in real time and transmitting the collected data to the master chip 1;

a pressure sensor 16 connected with the master chip 1 and used for sensing and collecting human body weight data as well as pressure data of a garment to a human body in real time and transmitting the collected data to the master chip 1;

a hydrogen sulfide sensor 17 connected with the master chip 1 and used for sensing and collecting concentration data of hydrogen sulfide discharged by the human body in real time and transmitting the collected data to the master chip 1;

a noise detection module 7 connected with the master chip 1 and used for monitoring the loudness of noise in the ambient environment;

a Bluetooth module 10 connected with the master chip 1 and used for sending data and alarm information; and

a power supply module 11 connected with the master chip 1 and used for providing a system power supply.

The master chip 1 is further connected with a voice alarm module 9 used for sending out an alarm signal. When the human body temperature and humidity data, the human body blood pressure data, the human body brain wave data, the human body heart rate data, the human body posture data and the hydrogen sulfide concentration data received by the master chip 1 exceed set values, the master chip 1 instantly instructs the voice alarm module 9 to send out an alarm signal.

The master chip 1 is further connected with a recording module 8 used for recording sounds in the ambient environment. When detecting that the noise, sensed by the noise detection module 7, in the ambient environment is greater than a set value, the master chip 1 instantly controls the recording module 8 to record the noise.

In order to better implement the scheme, the following optimization scheme is further provided:

Furthermore, the human body condition and behavior monitoring alarm system further comprises a GSM module 12 connected with the master chip 1 and used for sending alarm information to a mobile phone.

The GSM module 12 is provided with an SIM card holder, a GPRS module and a

GPS positioning module.

Furthermore, the human body condition and behavior monitoring alarm system further comprises a WIFI module 13 connected with the master chip 1 and used for sending alarm information to an external routing device.

Furthermore, the human body condition and behavior monitoring alarm system further comprises a touch screen 14 connected with the master chip 1 and used for displaying the system time and operating the system.

Furthermore, the human body condition and behavior monitoring alarm system further comprises an electronic stethoscope 18 connected with the recording module 8 and used for acquiring human body sound data.

The human body sound data acquired by the electronic stethoscope 18 are transmitted to the recording module 8 so as to be recorded.

Furthermore, the display 2 is an OLED display screen.

Furthermore, the master chip 1 is an STM32 master chip.

Furthermore, the temperature and humidity sensor 3, the blood pressure sensor 4, the brain wave sensor 5, the electronic gyroscope 6, the heart rate sensor 15, the pressure sensor 16 and the hydrogen sulfide sensor 17 are all sleeved with waterproof shells.

Furthermore, the human body condition and behavior monitoring alarm system further comprises a camera 19 connected with the master chip 1 and used for recording images of the ambient environment.

Furthermore, the noise detection module 7 is provided with a microphone 20.

Furthermore, the voice alarm module 9 is provided with a speaker 21.

Furthermore, the human body condition and behavior monitoring alarm system further comprises a solar cell 22 connected with the master chip 1 and used for providing a system power supply.

Furthermore, the human body condition and behavior monitoring alarm system further comprises a radio module 23 connected with the master chip 1.

Although the utility model is illustrated and explained through the specific embodiment and substitutes of the specific embodiment, it would appreciate that various modifications and changes made without deviating from the spirit scope of the utility model can also be implemented. Thus, the utility model is not limited in any sensor except by the claims and equivalent conditions of the claims.

1. A human body condition and behavior monitoring alarm system, characterized by comprising:
a master chip (1) used for receiving, sending and processing information and sending alarm commands;
a display (2) connected with the master chip (1) and used for displaying information data;
a temperature and humidity sensor (3) connected with the master chip (1) and used for sensing and collecting human body temperature and humidity data in real time and transmitting the collected data to the master chip (1);
a blood pressure sensor (4) connected with the master chip (1) and used for sensing and collecting human body blood pressure data in real time and transmitting the collected data to the master chip (1);
a brain wave sensor (5) connected with the master chip (1) and used for sensing and collecting human body brain wave data in real time and transmitting the collected data to the master chip (1);
an electronic gyroscope (6) connected with the master chip (1) and used for sensing and collecting human body posture data in real time and transmitting the collected data to the master chip (1);
a heart rate sensor (15) connected with the master chip (1) and used for sensing and collecting human body heart rate data in real time and transmitting the collected data to the master chip (1);
a pressure sensor (16) connected with the master chip (1) and used for sensing and collecting human body weight data as well as pressure data of a garment to a human body in real time and transmitting the collected data to the master chip (1);
a hydrogen sulfide sensor (17) connected with the master chip (1) and used for sensing and collecting concentration data of hydrogen sulfide discharged by the human body in real time and transmitting the collected data to the master chip (1);
a noise detection module (7) connected with the master chip (1) and used for monitoring a loudness of noise in an ambient environment;
a Bluetooth module (10) connected with the master chip (1) and used for sending data and alarm information; and
a power supply module (11) connected with the master chip (1) and used for providing a system power supply;
wherein, the master chip (1) is further connected with a voice alarm module (9) used for sending out an alarm signal, and when the human body temperature and humidity data, the human body blood pressure data, the human body brain wave data, the human body heart rate data, the human body posture data and the hydrogen sulfide concentration data received by the master chip (1) exceed set values, the master chip (1) instantly instructs the voice alarm module (9) to send out an alarm signal;
the master chip (1) is further connected with a recording module (8) used for recording sounds in the ambient environment; and when detecting that noise sensed by the noise detection module (7) in the ambient environment is greater than a set value, the master chip (1) instantly controls the recording module (8) to record the noise.

2. The human body condition and behavior monitoring alarm system according to claim 1, characterized by further comprising a GSM module (12) connected with the master chip (1) and used for sending alarm information to a mobile phone,
wherein, the GSM module (12) is provided with an SIM card holder, a GPRS module and a GPS positioning module.

3. The human body condition and behavior monitoring alarm system according to claim 1, characterized by further comprising a WIFI module (13) connected with the master chip (1) and used for sending alarm information to an external routing device.

4. The human body condition and behavior monitoring alarm system according to claim 1, characterized by further comprising a touch screen (14) connected with the master chip (1) and used for operating the system.

5. The human body condition and behavior monitoring alarm system according to claim 1, characterized by further comprising an electronic stethoscope (18) connected with the recording module (8) and used for acquiring human body sound data,
wherein, the human body sound data acquired by the electronic stethoscope (18) are transmitted to the recording module (8) so as to be recorded.

6. The human body condition and behavior monitoring alarm system according to claim 1, wherein the display (2) is an OLED display screen.

7. The human body condition and behavior monitoring alarm system according to claim 1, wherein the temperature and humidity sensor (3), the blood pressure sensor (4), the brain wave sensor (5), the electronic gyroscope (6), the heart rate sensor (15), the pressure sensor (16) and the hydrogen sulfide sensor (17) are all sleeved with waterproof shells.

8. The human body condition and behavior monitoring alarm system according to claim 1, characterized by further comprising a camera (19) connected with the master chip (1) and used for recording images of the ambient environment, wherein the noise detection module (7) is provided with a microphone (20), and the voice alarm module (9) is provided with a speaker (21).

9. The human body condition and behavior monitoring alarm system according to claim 1, characterized by further comprising a radio module (23) connected with the master chip (1).

10. The human body condition and behavior monitoring alarm system according to claim 1, characterized by further comprising a solar cell (22) connected with the master chip (1) and used for providing a system power supply.
